SEQUENCE LISTING (27 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 5 aa, Artificial Sequence
  CDR1 HC for Ipilimumab
SYTMH

SEQ ID NO: 4 - protein, 17 aa, Artificial Sequence
  CDR2 HC for Ipilimumab
FISYDGNNKYYADSVKG

SEQ ID NO: 5 - protein, 9 aa, Artificial Sequence
  CDR3 HC for Ipilimumab
TGWLGPFDY

SEQ ID NO: 6 - protein, 12 aa, Artificial Sequence
  CDR1 LC for Ipilimumab
RASQSVGSSYLA

SEQ ID NO: 7 - protein, 7 aa, Artificial Sequence
  CDR2 LC for Ipilimumab
GAFSRAT

SEQ ID NO: 8 - protein, 9 aa, Artificial Sequence
  CDR3 LC for Ipilimumab
QQYGSSPWT

SEQ ID NO: 9 - protein, 118 aa, Artificial Sequence
  Ipilimumab-VH
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 10 - protein, 329 aa, Artificial Sequence
  Human IgG1 constant HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 447 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 448 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 14 - protein, 107 aa, Artificial Sequence [sequence omitted]

SEQ ID NO: 15 - protein, 215 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 6 aa, Artificial Sequence
  Spacer
QGQSGS

SEQ ID NO: 17 - protein, 13 aa, Artificial Sequence
  Masking Moiety - YV39
CRTQLYGYNLCPY

SEQ ID NO: 18 - protein, 13 aa, Artificial Sequence
  Cleavable Moiety - 2001
ISSGLLSGRSDNH

SEQ ID NO: 19 - protein, 13 aa, Artificial Sequence
  Cleavable Moiety - 2011
ISSGLLSGRSDNP

SEQ ID NO: 20 - protein, 13 aa, Artificial Sequence
  Cleavable Moiety - 2012
ISSGLLSGRSANP

SEQ ID NO: 21 - protein, 146 aa, Artificial Sequence
  YV39-2001 VL
CRTQLYGYNLCPYGGGSSGGSISSGLLSGRSDNHGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 22 - protein, 146 aa, Artificial Sequence
  YV39-2011 VL
CRTQLYGYNLCPYGGGSSGGSISSGLLSGRSDNPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 23 - protein, 146 aa, Artificial Sequence
  YV39-2012 VL
CRTQLYGYNLCPYGGGSSGGSISSGLLSGRSANPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 24 - protein, 259 aa, Artificial Sequence
  Spacer - YV39-2011 LC
QGQSGSCRTQLYGYNLCPYGGGSSGGSISSGLLSGRSDNPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 439 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0332251 A1
(43) Pub. Date: Oct. 30, 2025

(54) USE OF SUCROSE, MANNITOL AND GLYCINE TO REDUCE RECONSTITUTION TIME OF HIGH CONCENTRATION LYOPHILIZED BIOLOGICS DRUG PRODUCTS

(71) Applicant: BRISTOL-MYERS SQUIBB COMPANY, Princeton, NJ (US)

(72) Inventors: Dilbir Singh BINDRA, Hillsborough, NJ (US); Barton J. DEAR, Martinsville, NJ (US); Yue HU, West Windsor, NJ (US); Ismahr-ehl MONDAL SIERRA, Kendall Park, NJ (US); Haresh Tukaram MORE, Mountainside, NJ (US); Duohai PAN, Princeton, NJ (US); Yongmei WU, East Brounswick, NJ (US)

(21) Appl. No.: 18/572,479

(22) PCT Filed: Jun. 17, 2022
(86) PCT No.: PCT/US2022/033958

§ 371 Date: Dec. 20, 2023

Related U.S. Application Data
(60) Provisional application No. 63/213,026, filed on Jun. 21, 2021.

(51) Int. Cl. A61K 39/395; A61K 9/19; A61K 47/18; A61K 47/26; C07K 16/28

(52) U.S. Cl. CPC A61K 39/3955; A61K 9/19; A61K 39/39591; A61K 47/183; A61K 47/26; C07K 16/2818; C07K 2319/00

(57) ABSTRACT

The present invention provides methods of lyophilizing proteins, including activatable antibodies such as an activatable ipilimumab, as well as related solution and lyophilized antibody formulations. Exemplary lyophilized formulations comprise a combination of mannitol and sucrose, in a weight ratio of two or three, or a combination of glycine and sucrose, in a weight ratio or two or three. Such lyophilized formulations exhibit stability and reduced reconstitution time.

### CROSS REFERENCE TO RELATED APPLICATIONS

This application claims priority to U.S. Provisional Application No. 63/213,026, filed Jun. 21, 2021, the disclosure of which is incorporated herein by reference.

### SEQUENCE LISTING

The Sequence Listing filed electronically herewith is also hereby incorporated by reference in its entirety (File Name: 20220603_SEQL_13503WOPCT_GB.txt; Date Created: 3 Jun. 2022; File Size: 38 KB).

### FIELD OF THE INVENTION

The present application discloses methods and formulations for lyophilizing therapeutic proteins, such as antibodies.

### BACKGROUND OF THE INVENTION

The immune system is capable of controlling tumor development and mediating tumor regression. This requires the generation and activation of tumor antigen-specific T cells. Multiple T-cell co-stimulatory receptors and T-cell negative regulators, or co-inhibitory receptors, act in concert to control T-cell activation, proliferation, and gain or loss of effector function. Among the earliest and best characterized T-cell co-stimulatory and co-inhibitory molecules are CD28 and CTLA-4. Rudd et al. (2009) Immunol. Rev. 229:12. CD28 provides co-stimulatory signals to T-cell receptor engagement by binding to B7-1 and B7-2 ligands on antigen-presenting cells, while CTLA-4 provides a negative signal down-regulating T-cell proliferation and function. CTLA-4, which also binds the B7-1 (CD80) and B7-2 (CD86) ligands but with higher affinity than CD28, acts as a negative regulator of T-cell function through both cell autonomous (or intrinsic) and cell non-autonomous (or extrinsic) pathways. Intrinsic control of CD8 and CD4 T effector (T_{eff}) function is mediated by the inducible surface expression of CTLA-4 as a result of T-cell activation, and inhibition of T-cell proliferation and cytokine proliferation by multivalent engagement of B7 ligands on opposing cells. Peggs et al. (2008) Immunol. Rev. 224:141.

Anti-CTLA-4 antibodies, when cross-linked, suppress T cell function in vitro. Krummel & Allison (1995) J. Exp. Med. 182:459; Walunas et al. (1994) Immunity 1:405. Regulatory T cells (T_{regs}), which express CTLA-4 constitutively, control T_{eff} function in a non-cell autonomous fashion. T_{regs} that are deficient for CTLA-4 have impaired suppressive ability (Wing et al. (2008) Science 322:271) and antibodies that block CTLA-4 interaction with B7 can inhibit T_{reg} function (Read et al. (2000) J. Exp. Med. 192:295; Quezada et al. (2006) J. Clin. Invest. 116:1935). More recently, T_{effs} have also been shown to control T cell function through extrinsic pathways (Corse & Allison (2012) J. Immunol. 189:1123; Wang et al. (2012) J. Immunol. 189:1118). Extrinsic control of T cell function by T_{regs} and T_{effs} occurs through the ability of CTLA-4-positive cells to remove B7 ligands on antigen-presenting cells, thereby limiting their co-stimulatory potential. Qureshi et al. (2011) Science 332: 600; Onishi et al. (2008) Proc. Nat'l Acad. Sci. (USA) 105:10113. Antibody blockade of CTLA-4/B7 interactions is thought to promote Teff activation by interfering with negative signals transmitted by CTLA-4engagement; this intrinsic control of T-cell activation and proliferation can promote both T_{eff} and T_{reg} proliferation (Krummel & Allison (1995) J. Exp. Med. 182:459; Quezada et al. (2006) J. Clin. Invest. 116:1935). In early studies with animal models, antibody blockade of CTLA-4 was shown to exacerbate autoimmunity. Perrin et al. (1996) J. Immunol. 157:1333; Hurwitz et al. (1997) J. Neuroimmunol. 73:57. By extension to tumor immunity, the ability of anti-CTLA-4 to cause regression of established tumors provided a dramatic example of the therapeutic potential of CTLA-4 blockade. Leach et al. (1996) Science 271:1734.

Human antibodies to human CTLA-4, ipilimumab and tremelimumab, were selected to inhibit CTLA-4-B7 interactions (Keler et al. (2003) J. Immunol. 171:6251; Ribas et al. (2007) Oncologist 12:873) and have been tested in a variety of clinical trials for multiple malignancies. Hoos et al. (2010) Semin. Oncol. 37:533; Ascierto et al. (2011) J. Transl. Med. 9:196. Ipilimumab, which was first approved for the treatment of metastatic melanoma, has since been approved for use in other cancers, and is in clinical testing in yet other cancers. Hoos et al. (2010) Semin. Oncol. 37:533; Hodi et al. (2010) N. Engl. J. Med. 363:711; Pardoll (2012) Nat. Immunol. 13(12): 1129. In 2011, ipilimumab, which has an IgG1 constant region, was approved in the US and EU for the treatment of unresectable or metastatic melanoma based on an improvement in overall survival in a phase III trial of previously treated patients with advanced melanoma. Hodi et al. (2010) N. Engl. J. Med. 363:711. Tumor regressions and disease stabilization were frequently observed, but treatment with these antibodies has been accompanied by adverse events with inflammatory infiltrates capable of affecting a variety of organ systems. The severity and frequency of side effects from treatment with ipilimumab, which carries a black box warning of immune-mediated adverse reactions, and to an even greater extent when combined with nivolumab (OPDIVO®), limits the use of ipilimumab by many treating physicians.

Activatable forms of ipilimumab have been developed in which the light chain contains a masking moiety that interferes with binding to CTLA-4, but is preferentially released in the tumor microenvironment after cleavage by proteases that are more prevalent and/or active in tumors than in peripheral tissues. WO 18/085555. Such tumor-specific activation enables full CTLA-4 blocking activity in the tumor microenvironment, promoting anti-tumor immune response, while minimizing CTLA-4 blockade in normal tissue, where it would otherwise cause systemic toxicity. As a consequence, the activatable form results in an increased therapeutic index compared with the native parent molecule.

Although activatable CTLA-4 antibodies provides therapeutic benefits, the novel protease cleavable linkers present challenges with regard to formulation and stability not present with ipilimumab. Because of their reduced toxicity, activatable antibodies may be administered at higher doses, and the lability of the protease cleavable linkers may result in instability during storage. Known methods of formulating ipilimumab may not be adequate for delivery of large doses of activatable CTLA-4 antibodies and long term storage without unwanted cleavage and degradation. The need exists for high concentration, stable formulations of activatable anti-CTLA-4 antibodies, such as Activatable Ipilimumab, and methods of manufacturing such formulations.

### SUMMARY OF THE INVENTION

The present invention provides formulations, including lyophilized formulations, of an activatable antibody, such as Activatable Ipilimumab, comprising mannitol and sucrose. In some embodiments the weight ratio of mannitol to sucrose is approximately two, or is approximately three. In further embodiments the combination of mannitol and sucrose comprise approximately 8.5% of the formulation by weight when reconstituted. In one embodiment the ratio is 2:1 and mannitol is at approximately 313 mM and sucrose is at approximately 83 mM, such as 313 mM mannitol and 83 mM sucrose.

The present invention also provides formulations, including lyophilized formulations, of an activatable antibody, such as Activatable Ipilimumab, comprising glycine and sucrose. In some embodiments the weight ratio of glycine to sucrose is approximately two or is approximately three. In further embodiments the combination of glycine and sucrose comprise approximately 8.5% of the formulation by weight when reconstituted. In one embodiment the ratio is 2:1 and glycine is at approximately 760 mM and sucrose is at approximately 83 mM, such as 760 mM glycine and 83 mM sucrose.

In some embodiments, the mannitol/sucrose or glycine/sucrose formulations, or lyophilized formulation thereof, comprise an activatable antibody comprising cleavable moiety (CM) 2011 comprising the sequence of SEQ ID NO: 19. In one such embodiment the activatable antibody is Activatable Ipilimumab comprising a heavy chain comprising the heavy chain variable region sequence of SEQ ID NO: 9 and a light chain comprising the light chain variable region sequence of SEQ ID NO: 22.

In various formulations the mannitol/sucrose or glycine/sucrose formulations of activatable antibody, such as Activatable Ipilimumab, the formulation further comprises histidine (pH 5.5), polysorbate 80 (PS80) and diethylenetriaminepentaacetic acid

(DTPA), e.g. 20 mM histidine (pH 5.5), 0.05% PS80 and 50 μM DTPA.

In various embodiments the mannitol/sucrose or glycine/sucrose formulations of the present invention comprise Activatable Ipilimumab at approximately 50 mg/ml or 80 mg/ml.

In some, but not all, embodiments, the invention provides lyophilized formulations of an activatable antibody, such as Activatable Ipilimumab. In some embodiments the lyophilized formulations comprise a unit dosage formulation (UDF) for delivery of a flat dose of Activatable Ipilimumab, such as 1600 mg, 1200 mg, 800 mg, 600 mg or 400 mg.

In some embodiments for delivery of 800 mg flat dose, the UDF comprises approximately 856 mg of Activatable Ipilimumab, providing 0.7 ml of overfill so that it will be possible to withdraw the full 800 mg dose conveniently and safely. Such 800 mg UDF embodiments are typically reconstituted at 80 mg/ml in a volume of 10.7 ml. In some embodiments, more than one 800 mg UDF may be administered as one dose, such as use of two 800 mg UDFs to administer 1600 mg of Activatable Ipilimumab, or three 800 mg UDFs to administer 2400 mg of Activatable Ipilimumab. In some embodiments, the 800 mg UDF is lyophilized in a 25R vial, and is reconstituted to a final volume of 10.7 ml, e.g. by addition of 9.6 ml sterile water for injection (SWFI).

In some 800 mg UDF embodiments, sucrose is present at approximately 304 mg with approximately 610 mg mannitol or glycine. In various embodiments, the 800 mg UDF of the present invention reconstitutes to a substantially clear solution at 80 mg/ml in 10 minutes or less, e.g. 5 minutes or less, or even 2 minutes or less, at room temperature.

Such 800 mg UDFs may further comprise approximately 33.2 mg histidine, 5.35 mg PS80 and 210 μg DTPA. Some specific 800 mg UDF embodiments comprise approximately 856 mg Activatable Ipilimumab, 33.2 mg histidine, 304 mg sucrose, 610 mg mannitol or glycine, 5.35 mg PS80 and 210 μg DTPA, optionally in a 25R vial.

In some embodiments for delivery of 600 mg flat dose, the UDF comprises approximately 656 mg of Activatable Ipilimumab so that it will be possible to withdraw the full 600 mg dose conveniently and safely. Such 600 mg UDF embodiments are typically reconstituted at 80 mg/ml in a volume of 8.2 ml. In some embodiments, more than one 600 mg UDF may be administered as one dose, such as use of two 600 mg UDFs to administer 1200 mg of Activatable Ipilimumab, or three 600 mg UDFs to administer 1800 mg of Activatable Ipilimumab. In some embodiments, the 600 mg UDF is lyophilized in a 25R vial, and is reconstituted to a final volume of 8.2 ml, e.g. by addition of approximately 7.36 ml SWFI.

In some 600 mg UDF embodiments, sucrose is present at approximately 233 mg with approximately 468 mg mannitol or glycine. In various embodiments, the 600 mg UDF of the present invention reconstitutes to a substantially clear solution at 80 mg/ml in 10 minutes or less, e.g. 5 minutes or less, or even 2 minutes or less, at room temperature.

Such 600 mg UDFs may further comprise approximately 25.4 mg histidine, 4.1 mg PS80 and 161 μg DTPA. Some specific 600 mg UDF embodiments comprise approximately 656 mg Activatable Ipilimumab, 25.4 mg histidine, 233 mg sucrose, 468 mg mannitol or glycine, 4.1 mg PS80 and 161 μg DTPA, optionally in a 25R vial.

In other embodiments for delivery of 400 mg flat dose, the UDF comprises approximately 435 mg of Activatable Ipilimumab so that it will be practically possible to withdraw the full 400 mg dose conveniently and safely. Such 400 mg UDF embodiments are typically reconstituted at 50 mg/ml in a volume of 8.7 ml. In some embodiments, more than one 400 mg UDF may be administered as one dose, such as use of two 400 mg UDFs to administer 800 mg of Activatable Ipilimumab, three 400 mg UDFs to administer 1200 mg of Activatable Ipilimumab, of four 400 mg UDFs to administer 1600 mg of Activatable Ipilimumab. In some embodiments, the 400 mg UDF is lyophilized in a 20R vial, and is reconstituted to a final volume of 8.7 ml, e.g. by addition of approximately 7.8 ml SWFI.

In some 400 mg UDF embodiments, sucrose is present at approximately 247 mg with approximately 496 mg mannitol or glycine. In various embodiments, the 400 mg UDF of the present invention reconstitutes to a substantially clear solution at 50 mg/ml in 10 minutes or less, e.g. 5 minutes or less, or even 2 minutes or less, at room temperature.

Such 400 mg UDFs may further comprise approximately 27 mg histidine, 4.35 mg PS80 and 171 μg DTPA. Some specific 400 mg UDF embodiments comprise approximately 435 mg Activatable Ipilimumab, 27 mg histidine, 247 mg sucrose, 496 mg mannitol or glycine, 4.35 mg PS80 and 171 μg DTPA, optionally in a 20R vial.

In some embodiments for delivery of 1200 mg flat dose, the UDF comprises approximately 1280 mg of Activatable Ipilimumab, providing 1.0 ml of overfill so that it will be possible to withdraw the full 1200 mg dose conveniently and safely. Such 1200 mg UDF embodiments are typically reconstituted at 80 mg/ml in a volume of 16 ml. In some embodiments, more than one 1200 mg UDF may be administered as one dose, such as use of two 1200 mg UDFs to administer 2400 mg of Activatable Ipilimumab. In some embodiments, the 1200 mg UDF is lyophilized in a 50 cc vial, and is reconstituted to a final volume of 16 ml, e.g. by addition of 14.3 ml SWFI.

In some 1200 mg UDF embodiments, sucrose is present at approximately 455 mg with approximately 912 mg mannitol or glycine. In various embodiments, the 1200 mg UDF of the present invention reconstitutes to a substantially clear solution at 80 mg/ml in 10 minutes or less, e.g. 5 minutes or less, or even 2 minutes or less, at room temperature.

Such 1200 mg UDFs may further comprise approximately 49.6 mg histidine, 8.0 mg PS80 and 314 μg DTPA. Some specific 1200 mg UDF embodiments comprise approximately 1280 mg Activatable Ipilimumab, 49.6 mg histidine, 455 mg sucrose, 912 mg mannitol or glycine, 8.0 mg PS80 and 314 μg DTPA, optionally in a 50 cc vial.

In some embodiments for delivery of 1600 mg flat dose, the UDF comprises approximately 1680 mg of Activatable Ipilimumab, providing 1.0 ml of overfill so that it will be possible to withdraw the full 1600 mg dose conveniently and safely. Such 1600 mg UDF embodiments are typically reconstituted at 80 mg/ml in a volume of 21 ml. In some embodiments, the 1600 mg UDF is lyophilized in a 50 cc vial, and is reconstituted to a final volume of 21 ml, e.g. by addition of 18.8 ml SWFI.

In some 1600 mg UDF embodiments, sucrose is present at approximately 597 mg with approximately 1197 mg mannitol or glycine. In various embodiments, the 1600 mg UDF of the present invention reconstitutes to a substantially clear solution at 80 mg/ml in minutes or less, e.g. 5 minutes or less, or even 2 minutes or less, at room temperature.

Such 1600 mg UDFs may further comprise approximately 65.2 mg histidine, 10.5 mg PS80 and 412 μg DTPA. Some specific1600 mg UDF embodiments comprise approximately 1680 mg Activatable Ipilimumab, 65.2 mg histidine, 597 mg sucrose, 1197 mg mannitol or glycine, 10.5 mg PS80 and 412 μg DTPA, optionally in a 50 cc vial.

In some embodiments, lyophilized formulations of the activatable antibody, such as Activatable Ipilimumab, of the present invention are packaged in a format selected from the group consisting of vials, ampules, prefilled syringes and autoinjectors. In some embodiments, such lyophilized formulations are included in kits comprising instructions for use. In some embodiments, the lyophilized formulation of Activatable Ipilimumab of the present invention requires no more than 8-16 minutes to fully dissolve the cake, such as no more than ten minutes or no more than five minutes.

In some embodiments, the lyophilized activatable antibodies, such as Activatable Ipilimumab, of the invention are stored in vials sealed under a vacuum, such as 500 mTorr.

In another aspect, the invention provides methods of lyophilizing activatable antibodies, such as Activatable Ipilimumab, comprising the steps of i) chilling filled vial to 5° C., and optionally holding them for 2 h followed by chilling the filled vials at −5° C. for 2 h; ii) freezing the pre-lyophilization solution at −40° C. for 180 minutes; iii) annealing at −10° C. for 5 h; iv) second freezing at −40° C. for 180 minutes; v) primary drying at −4° C. to −16° C., such as −13° C., at 100 mTorr or 150 mTorr for 58 h, or optionally primary drying at −9° C. at 150 mTorr for 83.3 h (for 20R and 25R vials) or at 100 mTorr for 90 h (for 50 cc vials); vi) secondary drying at 25° C. at 100 mTorr for 500 minutes; and vii) stoppering 5° C. under nitrogen at 720 torr. All temperature shifts are performed at 0.25° C./min to 0.5° C./min, and recited step times exclude time taken for temperature shift. Some embodiments include an annealing step, such as annealing for 3 h or 5 h, such as 5 h.

The invention further provides methods for preparing lyophilized activatable antibodies of the present invention, such as lyophilized unit dose formulations of activatable antibodies, comprising lyophilizing the activatable antibody in a lyophilization process comprising an annealing step, such as annealing for 3 h or 5 h, such as 5 h. Such methods may further include sealing the lyophilized formulation, including a unit dose formulation, in a vessel, such as a vial, under vacuum. In some embodiments, the vacuum is approximately or exactly 500 mTorr.

### BRIEF DESCRIPTION OF THE DRAWINGS

FIGS. 1A-1D illustrate exemplary product images for lyophilized Activatable Ipilimumab (“CTLA-4 PB” or “CTLA4 PB”) formulations of the present invention, showing vials containing unit doses after reconstitution. FIG. 1A: An 800 mg dose may be provided, e.g., in two 20R vials containing 8 ml (deliverable) of Activatable Ipilimumab at 50 mg/ml, or in a single 25R vial containing 10 ml (deliverable) of Activatable Ipilimumab at 80 mg/ml. FIG. 1B: A 1200 mg dose may be provided, e.g., in three 20R vials containing 8 ml (deliverable) of Activatable Ipilimumab at 50 mg/ml, in two 25R vials containing 7.5 ml (deliverable) of Activatable Ipilimumab at 80 mg/ml, or a single 50 cc vial containing 15 ml (deliverable) of Activatable Ipilimumab at 80 mg/ml. FIG. 1C: A 1600 mg fixed dose may be provided e.g., in four 20R vials containing 8 ml (deliverable) of Activatable Ipilimumab at 50 mg/ml, in two 25R vial containing 10 ml (deliverable) of Activatable Ipilimumab at 80 mg/ml, or a single 50 cc vial containing 20 ml (deliverable) of Activatable Ipilimumab at 80 mg/ml. FIG. 1D: A 2400 mg fixed dose may be provided e.g., in three 25R vials containing 10 ml (deliverable) of Activatable Ipilimumab at 80 mg/ml, or in two 50 cc vials containing 15 ml (deliverable) of Activatable Ipilimumab at 80 mg/ml. Fill volumes in the Figures do not include overfill. 20R and 25R vials typically include an additional 0.7 ml, and 50 cc vials typically contain and additional 1 ml, of reconstituted Activatable Ipilimumab as overfill to make it possible with withdraw the nominal sample volume. Vials for delivery of 600 mg of Activatable Ipilimumab at 80 mg/ml in a 25R vial are also provided herein, but are not illustrated.

FIG. 2 provides times, in seconds (s) or minutes (m), for solute dissolution and foam dissipation during reconstitution of lyophilized formulations of Activatable Ipilimumab of the present invention after storage for one month at 5° C., 25° C. and 40° C. See Example 3.

FIG. 3 provides measurements of stability for lyophilized formulations of Activatable Ipilimumab of the present invention after storage at different temperatures for one or two months. The lyophilized solution comprised 800 mg Activatable Ipilimumab, 20 mM histidine (pH 5.5), 83 mM sucrose, 313 mM mannitol, 0.05% PS80 and 50 μM DTPA. Lyophilized cakes were reconstituted at 80 mg/ml in SWFI after storage for the indicated time at the indicated temperature. Data are provided for percent intact, mono-clipped, and di-clipped mAb as measured by hydrophobic interaction chromatography (HIC); percent main peak, high MW and low MW as measured by size exclusion chromatography-high performance liquid chromatography (SEC-HPLC); and cumulative particle count at various particle sizes as detected using a HIAC® liquid particle counter instrument (Beckman Coulter Life Sciences, Indianapolis, Ind., USA). See Example 3.

FIG. 4 provides measurements of reconstitution time (referred to as “Cake disso.”) and stability for lyophilized formulations of Activatable Ipilimumab of the present invention after storage at different temperatures for one, three or six months. The lyophilized solution comprised 800 mg Activatable Ipilimumab, 20 mM histidine (pH 5.5), 83 mM sucrose, 313 mM mannitol, 0.05% PS80 and 50 μM DTPA. Lyophilized cakes were reconstituted at 80 mg/ml in SWFI after storage for the indicated time at the indicated temperature. Data are provided for percent intact, mono-clipped, and di-clipped mAb species as measured by size exclusion chromatography after proteolytic digestion with immunoglobulin-degrading enzyme of Streptococcus pyogenes (IdeS-SEC). An et al. (2014) Mabs 6:879. High MW and low MW species were measured by size exclusion chromatography-ultra performance liquid chromatography (SEC-UPLC); and acidic and basic species were measured by imaged capillary isoelectric focusing (icIEF), See Example 3.

FIGS. 5A and 5B provide front and top views, respectively, of reconstituted lyophilized Activatable Ipilimumab that had been stored in vials sealed at atmospheric pressure (left vial) and at 500 mTorr (right vial). Photographs were taken immediately after dissolution of the lyo cakes. See Example 4.

FIGS. 6A and 6B show lyophilized 25R vials for delivery of 800 mg and 600 mg of CTLA4-PB, respectively, produced as described at Example 5.

FIGS. 7A and 7B show 50 cc SGD vials for delivery of 1200 mg and 1600 mg of CTLA4-PB, respectively, produced as described at Example 6.

FIGS. 8A and 8B show lyophilized 25R vials for delivery of 800 mg of CTLA4-PB lyophilized in 1:1 and 3:1 Gly:Suc, respectively, produced as described at Example 7.

### DETAILED DESCRIPTION OF THE INVENTION

### Definitions

In order that the present disclosure may be more readily understood, certain terms are first defined. As used in this application, except as otherwise expressly provided herein, each of the following terms shall have the meaning set forth below. Additional definitions are set forth throughout the application.

“Activatable antibodies,” as used herein, refers to modified forms of antibodies that bind to targets of therapeutic interest wherein the antibodies comprise structural modifications that inhibit binding to the target until cleaved by proteases more prevalent and/or active in the tumor microenvironment than in peripheral tissue. “Activatable antibodies” encompasses activatable forms of anti-CTLA-4 antibody ipilimumab, such as antibodies comprising light chains modified to comprise a masking moiety (MM) and a cleavable moiety (CM), as disclosed in WO 18/085555, for example, Activatable Ipilimumab.

“Activatable Ipilimumab,” as used herein, refers to an activatable form of ipilimumab comprising a heavy chain comprising the heavy chain variable region sequence of SEQ ID NO: 9 and a light chain comprising the light chain variable region sequence selected from the group consisting of SEQ ID NOs: 21, 22 and 23. The light chain variable domain of an Activatable Ipilimumab may optionally further comprise a spacer of SEQ ID NO: 16 and the light chain may comprise a kappa constant domain of SEQ ID NO: 14, for example the spacer YV39-2011 light chain provided at SEQ ID NO: 24. The heavy chain of an Activatable Ipilimumab may further comprise an IgG1 constant domain of SEQ ID NO: 10, for example as in the ipilimumab heavy chain provided at SEQ ID NO: 11 or 12. Activatable Ipilimumab may comprise a heavy chain comprising SEQ ID NO: 11 or 12 and a light chain comprising a light chain of SEQ ID NO: 24.

“Adjuvant,” as used herein, refers to an agent that is administered to a subject in conjunction with a vaccine to enhance the immune response to the vaccine compared with the immune response that would result from administration of the vaccine without the adjuvant. Adjuvant may also refer to use of an agent after surgical removal of a tumor to reduce the risk of disease recurrence, such as use of ipilimumab or Activatable Ipilimumab following surgical removal of a melanoma.

“Administering,” “administer” or “administration” refers to the physical introduction of a composition comprising a therapeutic agent to a subject, using any of the various methods and delivery systems known to those skilled in the art. Preferred routes of administration for antibodies of the invention include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody of the invention can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

Unless otherwise indicated, administration of antibodies for the treatment of cancer is parenteral, such as intravenous (iv) or subcutaneous (sc). Methods of dosing and administration of the present invention can be performed for any number of cycles of treatment, from one, two, three, four cycles, etc., up to continuous treatment (repeating the dosing until no longer necessary, disease recurrence, or unacceptable toxicity is reached). For the purposes of the present disclosure, one cycle comprises the minimal unit of administration that includes at least one dose of each component (drug) of the combination therapy.

“Approximately,” as used herein with respect to amounts and concentrations of components of the various formulations herein, refers to ranges of values typically obtained in pharmaceutical formulations, such as amounts and concentrations within manufacturing tolerances. The degree of batch-to-batch variation that is considered within tolerances of the desired numerical (“nominal”) amount or concentration defines what is “approximately” the nominal amount or concentration. An “equivalent” amount or concentration, in contrast, refers to an amount or concentration that is not the same or approximately the same as a given nominal amount or concentration but is functionally equivalent to that amount or concentration with regard to stability of the activatable antibody in the formulation and the time for reconstitution from lyophilized form to a substantially clear solution.

“Initial Dose” or “initial dosing” as used herein refers to the first dosing of a patient with the regimen, and any subsequent repetitions of that same dosing regimen (such as second, third and fourth cycles, etc.), and is contrasted with “maintenance dose” or “maintenance dosing,” which refers to subsequent doses administered over a longer period after the initial dose or doses, e.g. longer than three months up to several years, or even indefinitely. Maintenance dosing may optionally comprise less frequent dosing and/or lower dose than the initial dose.

“Combination therapy,” as used herein, refers to administration of two or more therapeutic agents in a coordinated treatment plan, in which the dose and dosing interval of a first component of the combination is based on the dose and dosing interval of a second component, to elicit an overall therapeutic benefit. It is not limited to any particular details of administration, and encompasses administration as a mixture of the components, administration as separate compositions, whether concurrent or sequential on a given day. Although combination therapy is most convenient when dosing schedules are the same or multiples of one another (e.g. Q4W and Q8W), it also encompasses administration on different days if dosing intervals do not align for any given cycle.

An “antibody” (Ab) shall include, without limitation, a glycoprotein immunoglobulin which binds specifically to an antigen and comprises at least two heavy chains (HC) and two light chains (LC) interconnected by disulfide bonds. Each heavy chain comprises a heavy chain variable region (abbreviated herein as V_{H}) and a heavy chain constant region. The heavy chain constant region comprises three domains, C_{H1}, C_{H2} and C_{H3}. Each light chain comprises a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains comprise a binding domain that interacts with an antigen.

As used herein, and in accord with conventional interpretation, an antibody that is described as comprising “a” heavy chain and/or “a” light chain refers to antibodies that comprise “at least one” of the recited heavy and/or light chains, and thus will encompass antibodies having two or more heavy and/or light chains. Specifically, antibodies so described will encompass conventional antibodies having two substantially identical heavy chains and two substantially identical light chains. Antibody chains may be substantially identical but not entirely identical if they differ due to post-translational modifications, such as C-terminal cleavage of lysine residues, alternative glycosylation patterns, etc.

When used with reference to activatable antibodies, the “light chain variable domain” may further comprise a masking moiety, a cleavable moiety, a spacer element and optionally other sequence elements as disclosed herein.

Unless indicated otherwise or clear from the context, an antibody defined by its target specificity (e.g. an “anti-CTLA-4 antibody”) refers to antibodies that can bind to its human target (i.e. human CTLA-4). Such antibodies may or may not bind to CTLA-4 from other species.

The immunoglobulin may derive from any of the commonly known isotypes, including but not limited to IgA, secretory IgA, IgG and IgM. The IgG isotype may be divided in subclasses in certain species: IgG1, IgG2, IgG3 and IgG4 in humans, and IgG1, IgG2a, IgG2b and IgG3 in mice. “Isotype” refers to the antibody class (e.g., IgM or IgG1) that is encoded by the heavy chain constant region genes. “Antibody” includes, by way of example, both naturally occurring and non-naturally occurring antibodies, including allotypic variants; monoclonal and polyclonal antibodies; chimeric and humanized antibodies; human or non-human antibodies; wholly synthetic antibodies; and single chain antibodies. Unless otherwise indicated, or clear from the context, antibodies disclosed herein are human IgG1 antibodies. IgG1 constant domain sequences include, but are not limited to, known IgG1 allotypic variants.

The term “monoclonal antibody” (“mAb”) refers to a preparation of antibody molecules of single molecular composition, i.e., antibody molecules whose primary amino acid sequences are identical or essentially identical, and which exhibit a single binding specificity and affinity for a particular epitope. Monoclonal antibodies may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

An “antibody fragment” refers to a portion of a whole antibody, generally including the “antigen-binding portion” (“antigen-binding fragment”) of an intact antibody which retains the ability to bind specifically to the antigen bound by the intact antibody and also retains the Fc region of an antibody mediating FcR binding capability.

“Antibody-dependent cell-mediated cytotoxicity” (“ADCC”) refers to an in vitro or in vivo cell-mediated reaction in which nonspecific cytotoxic cells that express FcRs (e.g., natural killer (NK) cells, macrophages, neutrophils and eosinophils) recognize antibody bound to a surface antigen on a target cell and subsequently cause lysis of the target cell. In principle, any effector cell with an activating FcR can be triggered to mediate ADCC.

“Cancer” refers a broad group of various diseases characterized by the uncontrolled growth of abnormal cells in the body. Unregulated cell division and growth divide and grow results in the formation of malignant tumors or cells that invade neighboring tissues and may also metastasize to distant parts of the body through the lymphatic system or bloodstream.

A “cell surface receptor” refers to molecules and complexes of molecules capable of receiving a signal and transmitting such a signal across the plasma membrane of a cell.

“Effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcyγR-mediated effector functions such as ADCC and antibody dependent cell-mediated phagocytosis (ADCP), and down-regulation of a cell surface receptor (e.g., the B cell receptor; BCR). Such effector functions generally require the Fc region to be combined with a binding domain (e.g., an antibody variable domain).

An “immune response” refers to a biological response within a vertebrate against foreign agents, which response protects the organism against these agents and diseases caused by them. The immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues.

An “immunomodulator” or “immunoregulator” refers to a component of a signaling pathway that may be involved in modulating, regulating, or modifying an immune response. “Modulating,” “regulating,” or “modifying” an immune response refers to any alteration in a cell of the immune system or in the activity of such cell. Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments of the disclosed invention, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

A “protein” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. The term “protein” is used interchangeable herein with “polypeptide.”

“Reconstitution,” as used herein, refers to the process of solubilizing a lyophilized pellet, such as a lyophilized activatable antibody formulation of the present invention. Reconstitution time may be expressed herein in terms of a period of minutes, such as ten minutes or less, five minutes or less, or two minutes or less, or equivalently within a time interval, such as within ten minutes, within five minutes or within two minutes. Pursuant to United States Pharmacopeia (USP) guidance, the reconstitution endpoint is when the lyophilized solid dissolves completely, leaving no visible residue as undissolved matter, and the reconstituted solution is not significantly less clear than an equal volume of diluent in a similar vessel when examined similarly. United States Pharmacopeia Convention (2007) USP 31; The National Formulary: NF 26 at INJECTIONS: Constituted Solutions: Completeness and Clarity of Solution. Accordingly, reconstitution time as used herein refers to the time needed for the formation of a substantially clear solution phase that is not significantly less clear than an equal volume of SWFI, notwithstanding any foam that may remain in the vial.

A “subject” includes any human or non-human animal. The term “non-human animal” includes, but is not limited to, vertebrates such as nonhuman primates, sheep, dogs, rabbits, rodents such as mice, rats and guinea pigs, avian species such as chickens, amphibians, and reptiles. In preferred embodiments, the subject is a mammal such as a nonhuman primate, sheep, dog, cat, rabbit, ferret or rodent. In more preferred embodiments of any aspect of the disclosed invention, the subject is a human. Unless otherwise indicated, a subject as referred to herein is a human. The terms “subject” and “patient” are used interchangeably herein.

A “substantially clear solution,” as used herein with respect to a solution of activatable antibody reconstituted from a lyophilized formulation thereof of the present invention, is a colorless aqueous solution without visible cloudiness or suspended particulates when viewed by the naked eye, and which is not significantly less clear than an equal volume of diluent in a similar vessel when examined similarly The presence of a foam layer above a substantially clear aqueous solution does not make the solution other than substantially clear. Such foam layer may be selectively left behind during withdrawal of the aqueous solution, e.g., from a vial, in preparation for administration to a patient.

“SWFI,” as used herein, refers to sterile water for injection.

“Treatment” or “therapy” of a subject refers to any type of intervention or process performed on, or administering an active agent to, the subject with the objective of reversing, alleviating, ameliorating, inhibiting, slowing down or prevent the onset, progression, development, severity or recurrence of a symptom, complication, condition or biochemical indicia associated with a disease.

### High Concentration Formulations of Activatable Antibodies, Such as Activatable Ipilimumab, and Lyophilized Forms Thereof

The only approved anti-CTLA-4 antibody, ipilimumab (YERVOY®), provides long-term survival in up to 25% of metastatic melanoma patients when administered at 3 mg/kg (metastatic melanoma) or 10 mg/kg (adjuvant melanoma), but treatment is often accompanied by toxicity. Activatable antibodies that are preferentially activated by tumor-associated proteases hold the promise of reducing peripheral toxicity at a given dose, allowing higher (and thus potentially more efficacious) doses for any given level of toxicity, or some intermediate trade-off of the two. Activatable Ipilimumab has been proposed as an improved, safer way to target the CTLA-4 pathway than ipilimumab, which is known to cause limiting side-effects at higher doses. WO 18/085555. Activatable Ipilimumab comprises two heavy chains and two light chains in a conventional bivalent IgG structure, albeit with additional sequence elements (including a masking moiety MM and a cleavable moiety CM) at the amino termini of the light chains. Since each CM can be cleaved independently, Activatable Ipilimumab can exist as a mixture of intact/uncleaved, mono-cleaved, and dual-cleaved forms.

Activatable antibodies have the advantage over conventional antibodies of reduced peripheral toxicity. Such reduced toxicity permits higher dosing to drive higher efficacy at the tumor site, where the antibody is selectively cleaved to a fully active form. Activatable antibodies may also be subject to unwanted cleavage during storage due to lability of the cleavable linker sequence, necessitating storage under conditions that minimize cleavage, such as in lyophilized form.

Generating a high dose of a lyophilized antibody presents unique challenges in formulation. Due to finite volume of conventional pharmaceutical vials, such as 20R and 25R vials, formulations of activatable antibody of the present invention must be reconstituted at high concentration. At the same time, reconstitution must be fast enough for convenient administration, ideally requiring no more than 8-16 minutes. Conventional lyophilized antibody formulations can take up to thirty minutes to reconstitute at high concentration, e.g. greater than 50 mg/ml. Ideally, a therapeutic dose should be contained in four or fewer vials, preferably two or fewer, or optimally a single vial, and should reconstitute to a substantially clear solution within ten minutes, or preferably within five minutes, and optimally two minutes or less.

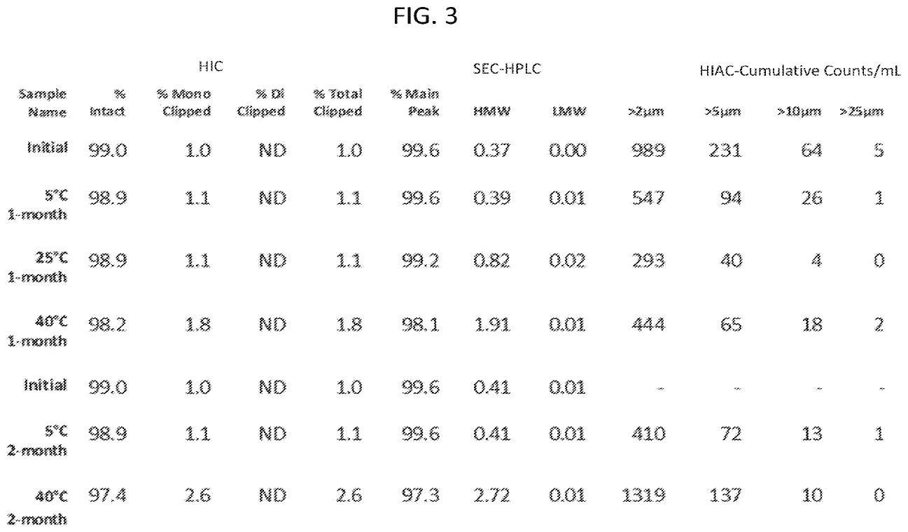

The lyophilized formulations provided herein, and related methods, enable long-term storage of activatable antibodies without undue cleavage or degradation by lyophilization, while also enabling rapid reconstitution at the high concentrations necessary to support high doses in a reasonable number of vials. Applicants have surprisingly found that addition of mannitol or glycine to protein formulations comprising sucrose significantly lowers the time needed to reconstitute a lyophilized cake at high concentration. Addition of mannitol or glycine at 1:1, 2:1 or 3:1 weight ratio to sucrose lowers dissolution times at 80 mg/ml from about 15 minutes down to only 2 to 4 minutes. See Example 2 and Table 2. See also FIG. 2. The combinations of 2:1 or 3:1 mannitol: sucrose and dissolution at 80 mg/ml rather than 100 mg/ml (or 120 mg/ml, not shown) was determined to be the optimal combination of high concentration and rapid dissolution for use in lyophilized formulations of Activatable Ipilimumab. Additional experiments confirmed that Activatable Ipilimumab was not degraded when stored in lyo cake form for at least a month, and up to six months, particularly at 4° C. or 25° C. See Example 3 and FIG. 3.

Although the rapid reconstitution of the lyophilized formulations of the present invention were illustrated with Activatable Ipilimumab, it is likely that reconstitution of any lyophilized protein would benefit from (be made faster by) the addition of mannitol or glycine at, e.g., 2:1 or 3:1 weight ratio with sucrose. Such lyophilized mannitol/sucrose or glycine/sucrose formulations would find the greatest in proteins that might otherwise be somewhat labile when stored as ready-to-use (RTU) solution form, such as activatable antibodies comprising a cleavage moiety such as 2011 (SEQ ID NO: 19). Rapid reconstitution at high concentration would be of still greater importance for antibodies to be delivered at high doses, since high concentration reconstitution minimizes the number of vials needed per dose. Activatable Ipilimumab has all of these features, and thus is perfectly suited for use in the lyophilized mannitol: sucrose and glycine: sucrose formulations of the present invention.

### Control of High Molecular Weight Aggregates

The lyophilized formulations provided herein, and related methods, further minimize formation of high molecular weight (HMW) species during storage and reconstitution. It was observed that the moisture content of the final product, surface area, and the amount of protein present on the void surface of lyophilized cake (“lyo cake”) all play important roles in product stability, particularly HMW formation upon storage. Moisture content of lyo cakes is maintained at <2% through the optimized lyophilization cycles of the present invention. Mannitol, the crystalline excipient in some embodiments of the formulations of the present invention, improves wettability of lyo cakes and helps reduce reconstitution time because of its presence on the void surface of lyo cake and in the amorphous matrix. However, due to phase separation of crystalline mannitol from amorphous phase, a small amount of protein is exposed without any protection from amorphous sucrose, leading to aggregation and formation of HMW species. Thus, it is critical to drive a proper balance for these different quality attributes.

The specific mannitol: sucrose ratio and lyophilization cycle parameters provided in the embodiments of the present invention mitigate moisture content, improve wettability and minimize HMW formation, keeping these critical quality attributes well within the limits set in the product specification.

Annealing, for example, was identified as a factor in optimizing the lyophilization of Activatable Ipilimumab. Lyophilization runs were conducted with no annealing, 3-hr and 5-hr annealing time. Results indicated that moisture content of the final product was higher in samples that did not go through an annealing step, and such higher moisture content may adversely affect product stability. See also Example 1.

Certain amino acids may also be used as stabilizers in the lyophilized formulations of the present invention. Amino acids are known to stabilize the protein through preferential exclusion from the protein-water interface in solution with volume exclusion effect similar to sugars. Timasheff (1992) “Stabilization of proteins structure by solvent additives,” in: T. Ahern, M. Manning (Eds.), Stability of Protein Pharmaceuticals, Part B: In Vivo Pathways of Degradation and Strategies for Protein Stabilization, Plenum, New York, pp. 265-285. It is known that amino acids demonstrate a general stabilizing effect on the long-term stability of sucrose based lyophilized products. Forney-Stevens et al. (2016) J. Pharm. Sci. 105:697. The hypothesis is that “the fit” of amino acids into the “free volume holes” of the amorphous formulation matrix is important in suppressing dynamics on a fast timescale, thereby leading to improved stability. Commonly used amino acids include hydrophobic amino acids, such as alanine, methionine, phenylalanine; uncharged polar amino acids, serine, threonine; and positively charged, such as lysine, histidine, arginine; and negatively charged, like aspartic acid and glutamic acid. In various additional embodiments of the present invention alanine, methionine or phenylalanine are added to the lyophilized formulation of activatable antibody to enhance stability.

### Formulations for Lyophilizing Activatable Ipilimumab

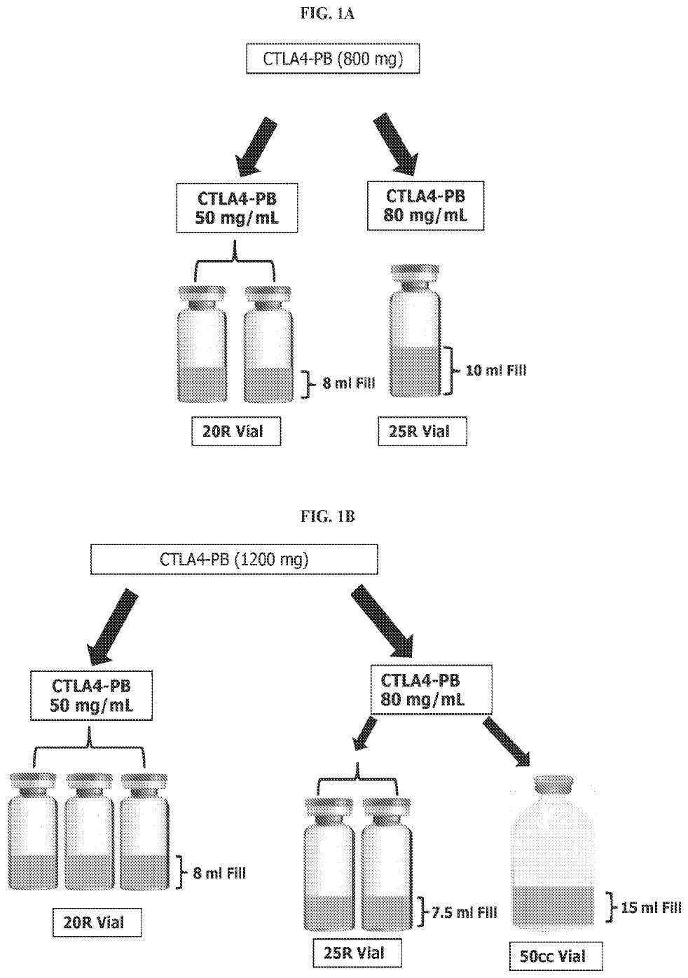

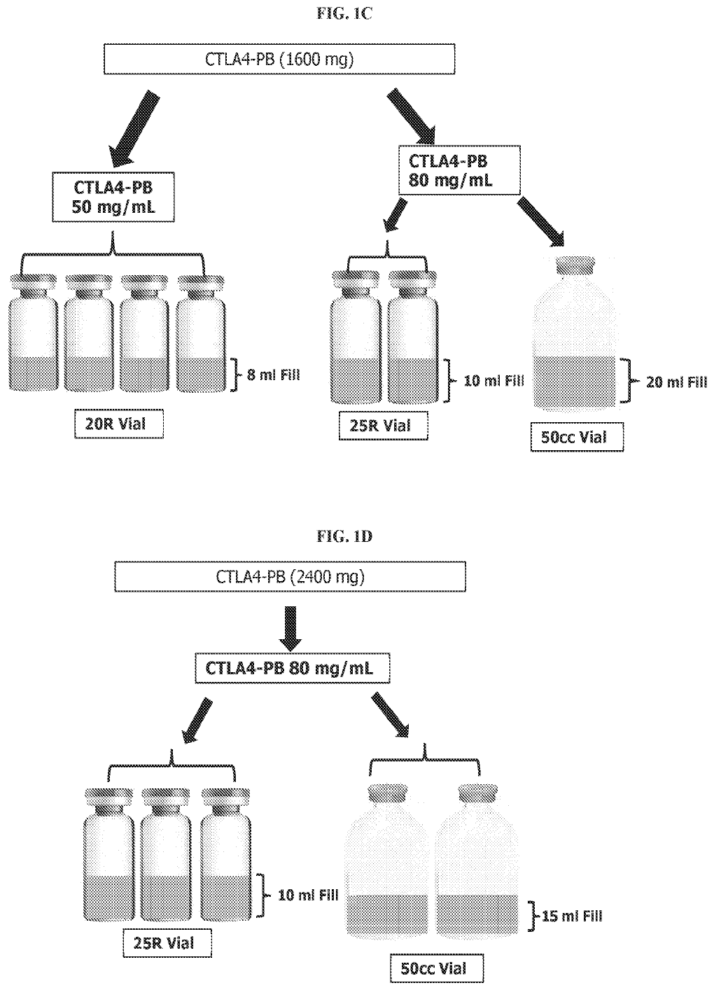

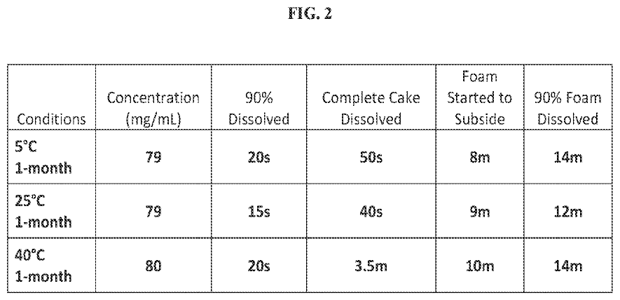

The present invention provides improved solution and lyophilized formulations of activatable antibodies, such as Activatable Ipilimumab. The formulations comprise sucrose and either mannitol or glycine, at 2:1 or 3:1 weight ratio, for example at a total weight concentration of 8.5% sucrose/mannitol or sucrose/glycine. Activatable antibodies, such as Activatable Ipilimumab, lyophilized in such formulations reconstitutes to a substantially clear solution at 50 to 80 mg/ml within a period of five minutes, such as within a period of two minutes, at room temperature. These high concentrations make it possible to provide fixed doses of up to 400 mg in a 20R vial at 50 mg/ml, 600 mg in a 25R vial at 80 mg/ml, 800 mg in a 25R vial at 80 mg/ml, and 1200 mg or 1600 mg in a 50 cc vial at 80 mg/ml. Some embodiments provide 0.7 ml of overfill and provide 435 mg of Activatable Ipilimumab in 8.7 ml in a 20R vial, 656 mg of Activatable Ipilimumab in 8.2 ml in a 25R vial, or 856 mg of Activatable Ipilimumab in 10.7 ml in a 25R vial. Other embodiments provide 1 ml of overfill and provide 1280 mg of Activatable Ipilimumab in 16 ml, or 1680 mg of Activatable Ipilimumab in 21 ml, in a 50 cc vial. Such unit dose vials enable deliverable doses of 400 mg, 600 mg, 800 mg, 1200 mg, 1600 mg, 1800 mg and 2400 mg in three or fewer vials. Exemplary product images for 800 mg, 1200 mg, 1600 mg and 2400 mg fixed doses of Activatable Ipilimumab are illustrated at FIGS. 1A-1D.

Additional excipients for use in the formulations of the present invention include histidine (pH 5.5), polysorbate 80 (PS80) and diethylenetriaminepentaacetic acid (DTPA), e.g. 20 mM histidine (pH 5.5), 0.05% PS80 and 50 μM DTPA.

In some embodiments lyophilized activatable antibodies of the present invention are lyophilized in siliconized vials, such as siliconized 20R or 25R vials, or in 50 cc SGD vials.

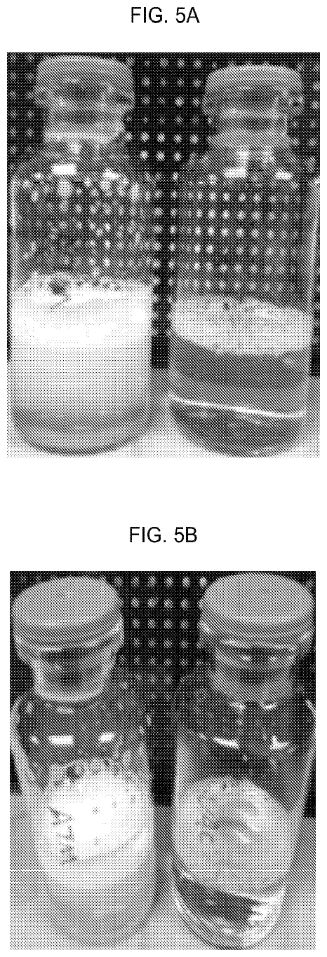

It has also been observed that reconstitution of lyophilized formulations can generate nanosized air bubbles that remain in solution for a long time. Zhou et al. (2016) J. Pharm. Sci. 105:2249. Snell et al. showed that the lyophilization of protein in presence of mannitol generates nanosize ice crystals during mannitol crystallization due to release of water. Snell et al. (2020) J. Pharm. Sci. 109:284. These nanosized ice crystals sublime during primary and secondary drying leaving nanosized voids, and upon reconstitution these voids creates nanobubbles. In the experiments described in Example 4, vials were stoppered under vacuum to minimize air bubble formation without affecting the quality of the product. When vials are stoppered under vacuum, these bubbles collapse immediately upon formation due to negative air pressure within the vial, as evidenced by FIGS. 5A and 5B. Accordingly, in some embodiments vials are sealed under negative pressure, such as at 500 mTorr rather than atmospheric pressure of 760Torr, to reduce foaming when diluent is added during reconstitution.

In various embodiments, diluent for reconstitution is sterile water for injection (SWFI). Reconstituted lyophilized formulations of the present invention may be further diluted into normal saline (NS) or 5% dextrose (D5W), e.g. in an infusion bag, in preparation for administration.

Exemplary embodiments and methods of the present invention are presented in the following examples.

### EXAMPLE 1

### Lyophilization of Activatable Ipilimumab

Activatable Ipilimumab was lyophilized in several formulations to determine which would best prevent degradation during storage and yet allow rapid reconstitution at high concentration. Exemplary formulations are provided at Table 1, in which the name of each formulation is provided across the top row, the components are listed in the left column, and the concentrations of the components in each formulation are provided in the appropriate cells.

TABLE 1
Formulations of Activatable Ipilimumab

| | Man:Suc | | Man:Suc | | Man:Suc | | Sucrose | | Arginine | | Gly:Suc | | | |
|---|---|---|---|---|---|---|---|---|---|---|---|---|---|---|
| | 1:1 | | 2:1 | | 3:1 | | (Control) | | 150 mM | | 2:1 | | PS80 1% | |
| Histidine | 20 | mM | 20 | mM | 20 | mM | 20 | mM | 20 | mM | 20 | mM | 20 | mM |
| (pH 5.5) | | | | | | | | | | | | | | |
| DTPA | 50 | μM | 50 | μM | 50 | μM | 50 | μM | 50 | μM | 50 | μM | 50 | μM |
| PS80 | 0.05% | | 0.05% | | 0.05% | | 0.05% | | 0.05% | | 0.05% | | 1.0% | |
| Sucrose | 125 | mM | 83 | mM | 62.5 | mM | 250 | mM | 250 | mM | 83 | mM | 250 | mM |

| | | | | | | | | | | |
|---|---|---|---|---|---|---|---|---|---|---|
| Mannitol | 235 | mM | 313 | mM | 353 | mM | — | — | — | — |

| | | | | | | | | |
|---|---|---|---|---|---|---|---|---|
| Arginine | — | — | — | — | 150 | mM | — | — |

| | | | | | | | | |
|---|---|---|---|---|---|---|---|---|
| Glycine | — | — | — | — | — | 760 | mM | — |

Lyophilization conditions were selected in part based on the glass transition temperature (T_{g}′) for the pre-lyophilized formulations, as measured by differential scanning calorimetry (DSC), and the collapse temperature (T_{c}) of the lyo cake, as measured by freeze-dry microscopy (FDM). A freezing temperature of −40° C. was chosen based on the glass transition temperatures for the formulations of Table 1, which ranged from −42° C. to −27° C. A primary drying temperature of −13° C. was selected based on the lack of a clear collapse temperature down to −10° C. to −15° C. for formulations with mannitol and glycine, respectively.

Based in part on these critical temperature concerns, formulations of Table 1 were lyophilized by a process essentially as follows: i) filled vials were chilled to 5° C., then held for 1 h at 5° C.; ii) pre-lyophilization solution was frozen at −40° C. for 180 minutes; iii) frozen solution was annealed at −10° C. for 5 h; iv) annealed solution was frozen a second time at −40° C. for 180 minutes; v) frozen samples were dried in a primary drying step at −13° C. at 100 mTorr for 58 h; vi) samples were then dried in a secondary drying step at 25° C. at 100 mTorr for 500 minutes; and vii) vials were stoppered at 5° C. under nitrogen. All temperature shifts were performed at 0.5° C./min, and recited step times exclude time taken for temperature shift to take place. No cake deformities were observed in any sample, which were typically white to off-white whole or fragmented cakes.

### EXAMPLE 2

### Reconstitution of Lyophilized Activatable Ipilimumab

Rapid reconstitution of the high concentration formulations of the present invention is important at the time of administration. Reconstitution time may be affected by such factors as cake wettability, porosity and crystallinity. Such properties of the lyophilized cake are, in turn, a function of variables including solution composition (e.g. the presence of mannitol and the ratio of mannitol to sucrose), the annealing step, the speed of the freezing step and the aggressiveness of the drying step (level of vacuum applied).

Exemplary lyophilized formulations of Activatable Ipilimumab were reconstituted to determine which formulation had the shortest reconstitution time. Reconstitution at room temperature was performed essentially as follows: i) the lyophilized sample vial was allowed to equilibrate to room temperature, ii) the exposed vial stopper was wiped with an alcohol pad; iii) the appropriate volume of SWFI was injected with a syringe and needle within 20 seconds (e.g. adding 9.6 ml of SWFI with a 10 ml silicon-free syringe with a 21 Gauge 1.5 inch needle to reconstitute 856 mg of Activatable Ipilimumab in 10.7 ml total volume, directing the water stream at the wall of the vial and not contacting the lyophilized cake with the needle tip) at which time a timer was started; iv) the vial was swirled gently for 30 seconds, taking care not to shake, at which point air bubbles were allowed to dissipate for 1 minute and the contents were then observed for completeness of dissolution; and v) step (iv) was repeated until the product was completely dissolved, as assessed by the absence of visible residue as undissolved matter, and the observation that the solution was not significantly less clear than an equal volume of diluent in a similar vessel when examined similarly, at which time the timer was stopped. Reconstituted solutions of the present invention were typically clear to slightly opalescent, and colorless to slightly yellow. Reconstituted solutions were briefly swirled again prior to sampling. Reconstitution times less than or equal to 30 seconds were reported as “<1 min”; greater than 30 seconds and less than or equal to 60 seconds were reported as “1 min”; and greater than 60 seconds were reported as the time in minutes rounded to the nearest minute.

Results for lyophilized Activatable Ipilimumab in the formulations of Table 1 are provided in Table 2.

TABLE 2
Reconstitution of Lyo Formulations of Activatable Ipilimumab

| | Man:Suc | | Man:Suc | | Man:Suc | | | | | | Gly:Suc | | | |
|---|---|---|---|---|---|---|---|---|---|---|---|---|---|---|
| | 1:1 | | 2:1 | | 3:1 | | Sucrose | | Arginine | | 2:1 | | PS80 1% | |
| Complete Cake | ~3-4 | min | ~3 | min | ~2 | min | ~16 | min | ~12 | min | ~2 | min | ~14 | min |
| Dissolution | | | | | | | | | | | | | | |
| 80 mg/ml | | | | | | | | | | | | | | |
| Complete Cake | 30 | min | 10-12 | min | 10-12 | min | 33 | min | — | | 12-13 | min | — | |
| Dissolution | | | | | | | | | | | | | | |
| 100 mg/ml | | | | | | | | | | | | | | |
| 90% Foam | 18-20 | min | 16-18 | min | 16-18 | min | 16 | min | 12 | min | 16-18 | min | 15-16 | min |
| Dissipation | | | | | | | | | | | | | | |
| 80 mg/ml | | | | | | | | | | | | | | |
| 90% Foam | 50 | min | 24 | min | 24 | min | — | | — | | 16 | min | — | |
| Dissipation | | | | | | | | | | | | | | |
| 100 mg/ml | | | | | | | | | | | | | | |

Both mannitol and glycine significantly reduced reconstitution time (Complete Cake Dissolution time) compared with other formulations that were tested. Reconstitution time is the time between addition of diluent (SWFI) and formation of a substantially clear lower phase from which nominal sample volume (in this case 10 ml from a vial reconstituted to 10.7 ml total volume) at the desired concentration (in this case 80 mg/ml) may be withdrawn. Pursuant to United States Pharmacopeia (USP) guidance, the substantially clear lower phase contains no visible residue as undissolved matter, and is not significantly less clear than an equal volume of diluent in a similar vessel when examined similarly. United States Pharmacopeia Convention (2007) USP 31; The National Formulary: NF 26 at INJECTIONS: Constituted Solutions: Completeness and Clarity of Solution. Regulatory specifications require reconstitution time of not greater than 20 minutes. Addition of both mannitol and glycine lead to dense foaming during reconstitution, and increased vial fogging, but did not interfere with formation of a substantially clear dissolved lower phase.

Mannitol and glycine containing formulations of the present invention improved reconstitution times at both 80 mg/ml and 100 mg/ml, but complete cake dissolution and reconstitution times were dramatically shorter at 80 mg/ml. Such rapid reconstitution, e.g. complete cake dissolution in no more than five minutes, is highly preferred in clinical medicine. The superior reconstitution and dissolution times at 80 mg/ml outweigh the incremental benefit of reconstitution at the higher concentration of 100 mg/ml.

### EXAMPLE 3

### Characterization of Solution Formulations of Activatable Ipilimumab

The stability of Activatable Ipilimumab in 2:1 and 3:1 Mannitol/Sucrose solution formulations was assessed over one month at 40° C., and over three months at 5° C. and 25° C., to determine whether the addition of mannitol at 2:1 or 3:1 affected stability in solution state, when measured by such parameters as percent intact, mono-clipped, and di-clipped mAb as measured by hydrophobic interaction chromatography (HIC); percent main peak, high MW and low MW as measured by size exclusion chromatography-high performance liquid chromatography (SEC-HPLC); and percent main, acidic and basic peak as measured by cation exchange chromatography (CEX). Both 2:1 and 3:1 Mannitol/Sucrose solution formulations showed identical stability results to a control formulation comprising sucrose at the same total weight percent mannitol and sucrose in the 2:1 and 3:1 formulations. This formulation is provided as the “sucrose” formulation of Table 1. Data not shown.

Reconstitution time and stability of Activatable Ipilimumab in 2:1 and 3:1 Mannitol/Sucrose lyophilized pellets were also assessed after storage for one month at 5° C., 25° C. and 40° C. and for two months at 5° C. and 40° C. The lyophilization solution comprised 80 mg Activatable Ipilimumab, 20 mM histidine (pH 5.5); 83 mM sucrose, 313 mM mannitol, 0.05% PS80 and 50 μM DTPA (Man:Suc 2:1 of Table 1). Lyophilized cakes were stored for the indicated time at the indicated temperature, and then reconstituted at 80 mg/ml in diluent essentially as described in Example 2. Reconstitution parameters for one month samples are provided at FIG. 2 which shows complete cake dissolution in less than five minutes for samples stored at all temperatures, and less than a minute when stored at 5° C. and 25° C.

Stability results for these reconstituted samples after one and two months are provided at FIG. 3. Activatable Ipilimumab showed less than 1% degradation when stored up to two months at 5° C. and 25° C. as measured by HIC and SEC-HPLC, and less than 3% degradation at 40° C. No changes were observed by capillary electrophoresis sodium dodecyl sulfate (CE-SDS) in any of the samples (data not shown).

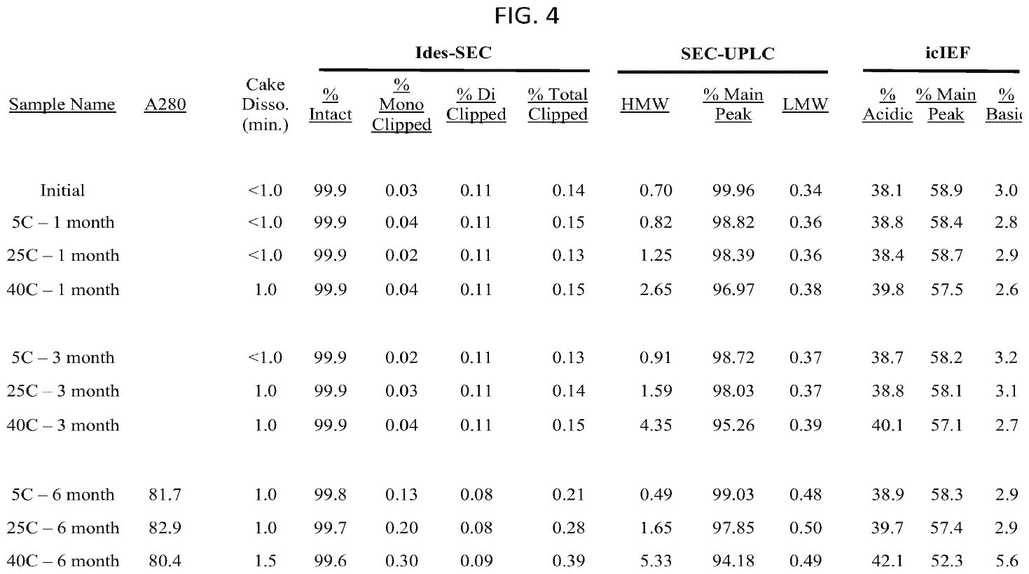

Additional stability and reconstitution data were collected after six months of storage at 5° C., 25° C., and 40° C. and are provided at FIG. 4. All samples reconstituted within 1 to 1.5 minutes with substantially full recovery of antibody as measured by absorption spectroscopy (A₂₈₀). Total clipped species remain less than 0.5% under all conditions. High molecular weight species remain less than 5% except under the most stringent conditions. The percentages of acidic and basic species remain substantially unchanged upon storage for six months, except at 40° C. where exhibit a small increase.

These results demonstrate that various formulations of the present invention can be used to generate stable lyophilized pellets of Activatable Ipilimumab that may be rapidly and conveniently reconstituted at the time of administration.

### EXAMPLE 4

### Reconstitution of Lyophilized Activatable Ipilimumab in Vials Sealed under Vacuum

Lyophilized pellets of Activatable Ipilimumab of the present invention, lyophilized essentially as described in Example 1, were reconstituted after storage for six months at 5° C., 25° C. or 40° C. Lyo cakes dissolved with rotating and shaking in 45-60 seconds (60-90 seconds for 40° C. samples), but bubbles persisted much longer, taking 45-60 minutes for 70% foam dissipation and 75-105 minutes for 90% foam dissipation. Although the presence of foam did not interfere with the ability to withdraw the intended volume of protein, or reduce the concentration of protein, its presence was undesirable because it interfered with the ability to determine visually when reconstitution was complete. Further experiments were performed to determine if storage under vacuum could reduce foaming.

Activatable Ipilimumab was lyophilized in 25R ISO vials, essentially as described in Example 1, and sealed under nitrogen at atmospheric pressure, or at 500 mTorr, and stored for 6 months at 5° C. Lyo cakes were then reconstituted in 9.4 ml of water with rotating and shaking. Lyo cakes fully dissolved after 45-60 seconds, at which point photographs were taken to illustrate the difference in the level of foam in vials sealed at atmospheric pressure and those sealed under vacuum. See FIGS. 5A and 5B. Vials sealed under vacuum exhibited significantly less foam.

### EXAMPLE 5

### Optimized Lyophilization Process to Reduce Vial Fogging

The process of lyophilization of Activatable Ipilimumab (CTLA4-PB) was optimized to reduce fogging of 25R vials essentially as follows.

Exemplary vials are for delivery of 600 mg of CTLA4-PB, reconstituted in 7.5 mL of water, at 80 mg/mL, including 0.7 mL of overfill for a total fill volume of 8.2 mL and total CTLA4-PB of 656 mg/vial. The lyo process comprises i) chilling the filled vials to 5° C. and holding them for 2 h, followed by chilling the filled vials at −5° C. for 2 h; ii) freezing the pre-lyophilization solution at −40° C. for 180 minutes; iii) annealing at −10° C. for 5 h; iv) second freezing at −40° C. for 180 minutes; v) primary drying at −9° C. at 100 mTorr for 50 h; vi) secondary drying at 25° C. at 100 mTorr for 500 minutes; and vii) stoppering at 5° C. under nitrogen at 720 torr. All temperature shifts are performed at 0.5° C./min.

Additional exemplary vials are for delivery of 800 mg of CTLA4-PB, reconstituted in 10 mL of water, at 80 mg/mL, including 0.7 mL of overfill for a total fill volume of 10.7 mL and total CTLA4-PB of 856 mg/vial. The lyo process comprises i) chilling the filled vials to 5° C. and holding them for 2 h followed by chilling the filled vials at −5° C. for 2 h; ii) freezing the pre-lyophilization solution at −40° C. for 180 minutes; iii) annealing at −10° C. for 5 h; iv) second freezing at −40° C. for 180 minutes; v) primary drying at −9° C. at 150 mTorr for 83.3 h; vi) secondary drying at 25° C. at 150 mTorr for 500 minutes; and vii) stoppering at 5° C. under nitrogen at 720 torr. All temperature shifts are performed at 0.5° C./min.

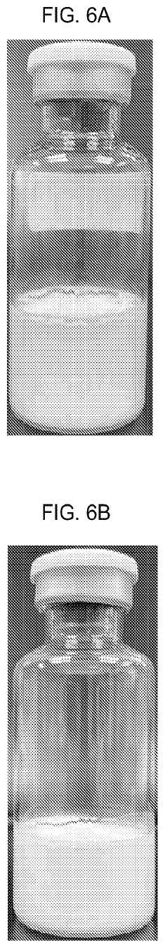

Holding vials at 5° C. and −5° C. shows significant improvement in minimizing the fogging, compared with lyophilization without these holds, with minimal to no fogging in lyophilized DP vials. Exemplary vials lyophilized by the methods of this example are provided at FIGS. 6A and 6B.

### EXAMPLE 6

### Lyophilization of Activable Ipilimumab in 50 cc Vials with Optimized Lyophilization Process to Reduce Fogging

The process of lyophilization of Activatable Ipilimumab (CTLA4-PB) was optimized to reduce fogging of 50 cc SGD vials essentially as follows.

Exemplary 50 cc vials are for delivery of 1200 mg of CTLA4-PB, reconstituted at 80 mg/mL in 14.3 mL of water, for a total volume after reconstitution of 16 mL (which includes 1 mL of overfill) and total CTLA4-PB of 1280 mg/vial. Additional exemplary 50 CC vials are for delivery of 1600 mg of CTLA4-PB, reconstituted at 80 mg/mL in 18.8 mL of water, for a total volume after reconstitution of 21 mL (which includes 1 mL of overfill) and total CTLA4-PB of 1680 mg/vial.

TABLE 3
50 cc Lyophilized Vials

| | Mannitol | Sucrose | Man:Suc | Fill volume | Vial Strength | |
|---|---|---|---|---|---|---|
| Formulation | (mg/mL) | (mg/mL) | (w/w) | (mL) | (mg/vial) | Vial size |
| CTLA-PB 80 | 57 | 28.4 | 2:1 | 21 | 800 | 50-cc |
| mg/mL | | | | 16 | 600 | SGD vial |

The lyo process comprises i) chilling the filled vials to 5° C. and holding them for 2 h followed by chilling the filled vials at −5° C. for 2 h; ii) freezing the pre-lyophilization solution at −40° C. for 180 minutes; iii) annealing at −10° C. for 5 h; iv) second freezing at −40° C. for 180 minutes; v) primary drying at −9° C. at 100 mTorr for 90 h; vi) secondary drying at 25° C. at 100 mTorr for 500 minutes; and vii) stoppering at 5° C. under nitrogen at 720 torr. All temperature shifts are performed at 0.5° C./min.

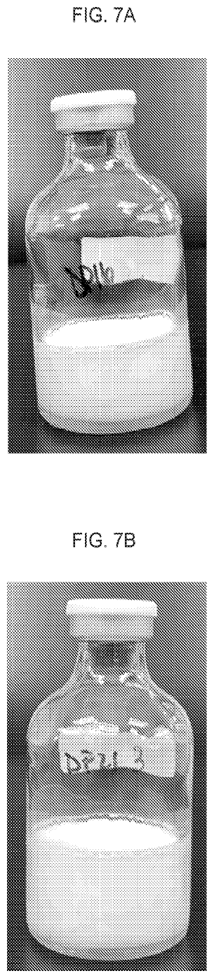

The lyophilized vials showed intact cake with no cracks or shrinkages. Minimal to no fogging was observed. Exemplary 50 cc vials lyophilized by the methods of this example are shown at FIGS. 7A and 7B. Moisture content analysis by near infrared analysis, reconstitution time, aggregation by SE-HPLC and particulate matter by HIAC results are shown in Tables 4-6.

TABLE 4
Moisture Content in 50 cc Lyophilized Vials

| | Average Residual | Reconstitution |
|---|---|---|
| Sample | Moisture (%) | Time (min) |
| CTLA4-PB 1600 mg/vial | 1.91 | ≈4 |
| (80 mg/mL, 21 mL Fill) | | |
| CTLA4-PB 1200 mg/vials | 1.79 | ≈3-4 |
| (80 mg/mL, 16 mL Fill) | | |

TABLE 5
SE-HPLC Aggregation Analysis of 50 cc Lyophilized Vials

| Sample | % LMW | % HMW | % Monomer |
|---|---|---|---|
| CTLA4-PB 1600 mg/vial | 0.1 | 1.0 | 96.9 |
| (80 mg/mL, 21 mL Fill) | | | |
| CTLA4-PB 1200 mg/vial | 0.1 | 1.0 | 96.9 |
| (80 mg/mL, 16 mL Fill) | | | |

TABLE 6
Particulate Matter by HIAC Analysis of 50 cc Lyophilized Vials

| | Cumulative Particles/ml | | | |
|---|---|---|---|---|
| Sample | ≥2 um | ≥5 um | ≥10 um | ≥25 um |
| CTLA4-PB 1600 mg/vial | 246 | 34 | 4 | 1 |
| (80 mg/mL, 21 mL Fill) | | | | |
| CTLA4-PB 1200 mg/vial | 205 | 28 | 2 | 0 |
| (80 mg/mL, 16 mL Fill) | | | | |

### EXAMPLE 7

### Lyophilization of Activatable Ipilimumab in Glycine: Sucrose

Activatable Ipilimumab was lyophilized in glycine:sucrose (“Gly:Suc”) at 1:1 and 3:1 w/w, essentially as follows. Exemplary 25R vials for delivery of 800 mg of CTLA4-PB were prepared as in Example 5, except they were lyophilized with glycine: sucrose, as described at Table 7.

TABLE 7
Gly:Suc Lyophilization of CTLA4-PB

| | | | | Fill | Vial | |
|---|---|---|---|---|---|---|
| | Glycine | Sucrose | Gly:Suc | volume | Strength | |
| Formulation | (mg/mL) | (mg/mL) | (w/w) | (mL) | (mg/vial) | Vial size |
| CTLA4-PB | 42.8 | 42.8 | 1:1 | 10.7 | 800 | 25R |
| 80 mg/mL | | | | | | |
| CTLA4-PB | 64.2 | 21.4 | 3:1 | 10.7 | 800 | 25R |
| 80 mg/mL | | | | | | |

The lyo process comprises i) chilling the filled vials to 5° C.; ii) freezing the pre-lyophilization solution at −40° C. for 180 minutes; iii) annealing at −10° C. for 5 h; iv) second freezing at −40° C. for 180 minutes; v) primary drying at −13° C., at 100 mTorr for 60 h; vi) secondary drying at 25° C. at 100 mTorr for 500 minutes; and vii) stoppering at 5° C. under nitrogen at 720 torr.] All temperature shifts are performed at 0.5° C./min

Moisture content analysis by near infrared analysis, reconstitution time, aggregation by SE-HPLC and particulate matter by HIAC results are shown in Tables 8-10.

TABLE 8
Moisture Content in Gly:Suc Lyophilized Vials

| | Average Residual | Reconstitution |
|---|---|---|
| Sample | Moisture (%) | Time (min) |
| CTLA4-PB 80 mg/mL (Gly:Suc | 1.17 | ≈4-5 |
| 1:1 w/w) | | |
| CTLA4-PB 80 mg/mL (Gly:Suc | 0.67 | ≈4-5 |
| 3:1 w/w) | | |

TABLE 9
SE-HPLC Aggregation Analysis of Gly:Suc Lyophilized Vials

| Sample | % LMW | % HMW | % Monomer |
|---|---|---|---|
| CTLA4-PB 80 mg/mL (Gly:Suc | 0.1 | 0.2 | 99.7 |
| 1:1 w/w) | | | |
| CTLA4-PB 80 mg/mL (Gly:Suc | 0.1 | 1.1 | 98.8 |
| 3:1 w/w) | | | |

TABLE 10
Particulate Matter by HIAC Analysis Gly:Suc Lyophilized Vials

| | Cumulative Particles/mL | | | |
|---|---|---|---|---|
| Sample | ≥2 μm | ≥5 μm | ≥10 μm | ≥25 μm |
| CTLA4-PB 80 mg/mL (Gly:Suc | 345 | 34 | 4 | 0 |
| 1:1 w/w) | | | | |
| CTLA4-PB 80 mg/mL (Gly:Suc | 332 | 28 | 4 | 0 |
| 3:1 w/w) | | | | |

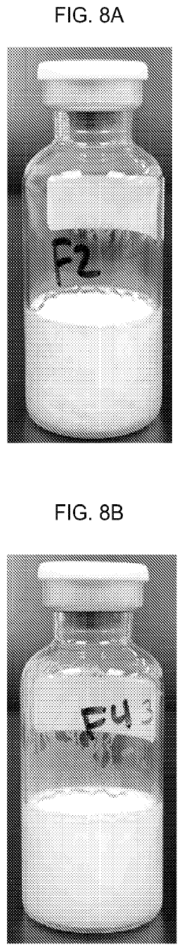

The lyophilized vials showed intact cake with no cracks or shrinkages. Minimal to no fogging was observed. Exemplary 25R vials lyophilized by the methods of this example, at Gly:Suc 1:1 and 3:1 w/w, are shown at FIGS. 8A and 8B, respectively. All lyophilized DP vials showed minimal to no fogging in formulation with Gly:Suc 1:1 w/w ratio. The CTLA4-PB formulation with Gly:Suc at 3:1 w/w ratio showed fogging in all vials.

### EXAMPLE 8

### Lyophilization of Activatable Ipilimumab with High Temperature Primary Drying with Optimized Lyophilization Process to Reduce Fogging

Activatable Ipilimumab was lyophilized in mannitol:sucrose 2:1 w/w and in glycine:sucrose 2:1 w/w at elevated primary drying temperature, essentially as follows. Exemplary 25R vials for delivery of 800 mg of CTLA4-PB were prepared as in Example 5, except they were lyophilized in mannitol: sucrose or glycine: sucrose, as described at Table 11.

TABLE 11
Mannitol:Sucrose and Glycine: Sucrose Lyophilization

| | | | | | Vial | |
|---|---|---|---|---|---|---|
| | Mannitol | Glycine | Sucrose | Fill volume | Strength | |
| Formulation | (mg/mL) | (mg/mL) | (mg/mL) | (mL) | (mg/vial) | Vial size |
| CTLA4-PB | 57.0 | — | 28.4 | 10.7 | 800 | 25R |
| 80 mg/mL | | | | | | |
| CTLA4-PB | — | 57.1 | 28.5 | 10.7 | 800 | 25R |
| 80 mg/mL | | | | | | |

Addition of mannitol or glycine as a crystalline bulking agent during lyophilization enables primary drying (PD) at shelf temperatures above 0° C. Therefore CTLA4-PB 80 mg/mL lyophilization was performed at primary drying shelf temperature at 10° C. Also, a single step drying was performed, at shelf temperature of 25° C.

The lyo process comprises i) chilling the filled vials to 5° C. and holding them for 2 h followed by chilling the filled vials at −5° C. for 2 h; ii) freezing the pre-lyophilization solution at −40° C. for 180 minutes; iii) annealing at −10° C. for 5 h; iv) second freezing at −40° C. for 180 minutes; v) primary drying at 10° C. at 100 mTorr for 38 h with secondary drying at 25° C. at 100 mTorr for 500 minutes, or single step drying at 25° C. at 100 mTorr for 36.3 h; and vii) stoppering at 5° C. under nitrogen at 720 torr. All temperature shifts are performed at 0.5° C./min.

The lyophilized vials showed intact cake with no cracks or shrinkages. Minimal to no fogging was observed in mannitol:sucrose 2:1 formulation vials at high shelf temperature. The glycine:sucrose 2:1 vials showed significant fogging at high shelf temperature. Moisture content analysis by near infrared analysis, reconstitution time, aggregation by SE-HPLC, and particulate matter by HIAC results are shown in Tables 12-16.

TABLE 12
Moisture Content in Lyophilized Vials
Dried at Elevated Temperatures

| | Primary Drying | Residual | |
|---|---|---|---|
| | Shelf | Moisture | Reconstitution |
| Sample | Temperature | (%) | Time (min) |
| CTLA4-PB 800 mg/mL | 10° C. | 1.25 | ≈3-4 |
| (Mannitol:Sucrose 2:1 w/w) | | | |
| CTLA4-PB 800 mg/mL | 25° C. | 1.74 | ≈3-4 |
| (Mannitol:Sucrose 2:1 w/w) | | | |
| CTLA4-PB 800 mg/mL | 10° C. | 0.71 | ≈3-4 |
| (Glycine:Sucrose 2:1 w/w) | | | |
| CTLA4-PB 800 mg/mL | 25° C. | 0.71 | ≈2-3 |
| (Glycine:Sucrose 2:1 w/w) | | | |

TABLE 13
SE-HPLC Aggregation Analysis of Mannitol:Sucrose 2:1
w/w Lyophilized Vials Dried at Elevated Temperatures

| | Primary Drying Shelf Temperature | | | Single step Drying Shelf Temperature | | |
|---|---|---|---|---|---|---|
| | (10° C.) | | | (25° C.) | | |
| Timepoint | % LMW | % HMW | % Monomer | % LMW | % HMW | % Monomer |
| Initial | 0.1 | 1.6 | 98.3 | 0.1 | 1.4 | 98.5 |
| 5° C. 3 months | 0.1 | 1.7 | 98.2 | 0.1 | 1.9 | 98.1 |
| 25° C. 3 months | 0.1 | 2.7 | 97.1 | 0.1 | 2.5 | 97.4 |
| 40° C. 3 months | 0.1 | 4.9 | 95.0 | 0.1 | 4.9 | 95.0 |
| 5° C. 6 months | 0.1 | 1.8 | 98.1 | 0.1 | 1.9 | 98.0 |
| 25° C. 6 months | 0.1 | 3.6 | 96.4 | 0.1 | 3.3 | 96.6 |

TABLE 14
SE-HPLC Aggregation Analysis of Glycine:Sucrose 2:1
w/w Lyophilized Vials Dried at Elevated Temperatures

| | Primary Drying Shelf | | | Single step Drying Shelf Temperature | | |
|---|---|---|---|---|---|---|
| | Temperature (10° C.) | | | (25° C.) | | |
| Timepoint | % LMW | % HMW | % Monomer | % LMW | % HMW | % Monomer |
| Initial | 0.1 | 0.9 | 99.0 | 0.1 | 0.9 | 99.0 |

TABLE 15
Particulate Matter by HIAC Analysis of Mannitol: Sucrose
2:1 w/w Lyophilized Vials Dried at Elevated Temperatures

| | Primary Drying Shelf Temperature | | | | Single step Drying Shelf Temperature | | | |
|---|---|---|---|---|---|---|---|---|
| | (10° C.) | | | | (25° C.) | | | |
| | Cumulative Particles/mL | | | | Cumulative Particles/mL | | | |
| Time point | ≥2 μm | ≥5 μm | ≥10 μm | ≥25 μm | ≥2 μm | ≥5 μm | ≥10 μm | ≥25 μm |
| Initial | 334 | 73 | 24 | 2 | 321 | 34 | 7 | 0 |
| 5 C., 6 months | 822 | 151 | 20 | 1 | 485 | 81 | 11 | 2 |
| 25 C., 6 months | 671 | 111 | 17 | 2 | 600 | 78 | 6 | 0 |

TABLE 16
Particulate Matter by HIAC Analysis of Glycine: Sucrose
2:1 w/w Lyophilized Vials Dried at Elevated Temperatures

| | Primary Drying Shelf Temperature | | | | Single step Drying Shelf Temperature | | | |
|---|---|---|---|---|---|---|---|---|
| | (10° C.) | | | | (25° C.) | | | |
| Time | Cumulative Particles/mL | | | | Cumulative Particles/mL | | | |
| point | ≥2 μm | ≥5 μm | ≥10 μm | ≥25 μm | ≥2 μm | ≥5 μm | ≥10 μm | ≥25 μm |
| Initial | 195 | 22 | 4 | 1 | 310 | 15 | 4 | 1 |

A summary of the sequence listing as provided at Table 17.

TABLE 17
Summary of the Sequence Listing

| SEQ ID NO. | Description |
|---|---|
| 1 | human CTLA-4 (NP_005205.2) |
| 2 | human CD28 (NP_006130.1) |
| 3 | ipilimumab CDRH1 |
| 4 | ipilimumab CDRH2 |
| 5 | ipilimumab CDRH3 |
| 6 | ipilimumab CDRL1 |
| 7 | ipilimumab CDRL2 |
| 8 | ipilimumab CDRL3 |
| 9 | ipilimumab heavy chain variable domain |
| 10 | IgG1 constant domain |
| 11 | ipilimumab heavy chain lacking C-terminal K |
| 12 | ipilimumab heavy chain |
| 13 | ipilimumab light chain variable domain |
| 14 | kappa constant domain |
| 15 | ipilimumab light chain |
| 16 | Spacer QGQSGS |
| 17 | masking moiety YV39 |
| 18 | cleavable moiety 2001 |
| 19 | cleavable moiety 2011 |
| 20 | cleavable moiety 2012 |
| 21 | YV39-2001 VL |
| 22 | YV39-2011 VL |
| 23 | YV39-2012 VL |
| 24 | Spacer YV39-2011 LC |
| 25 | nivolumab heavy chain lacking C-terminal K |
| 26 | nivolumab heavy chain |
| 27 | nivolumab light chain |

With regard to antibody sequences, the Sequence Listing provides the sequences of the mature variable regions and heavy and light chains, i.e. the sequences do not include signal peptides.

### EQUIVALENTS

Those skilled in the art will recognize, or be able to ascertain using no more than routine experimentation, many equivalents of the specific embodiments disclosed herein. Such equivalents are intended to be encompassed by the following claims.

1. A formulation of an activatable antibody comprising:
a. sucrose; and
b. mannitol or glycine.

2. The formulation of activatable antibody of claim 1 wherein the activatable antibody comprises a cleavable moiety comprising the sequence of SEQ ID NO: 19.

3. The formulation of activatable antibody of claim 2 wherein the activatable antibody is Activatable Ipilimumab comprising:
a. a heavy chain comprising the heavy chain variable region sequence of SEQ ID NO: 9; and
b. a light chain comprising the light chain variable region sequence of SEQ ID NO: 22.

4. The formulation of an activatable antibody of claim 3 comprising:
a. 83 mM sucrose; and
b. 313 mM mannitol or approximately 760 mM glycine.

5. The formulation of activatable antibody of claim 4 further comprising:
a. histidine (pH 5.5);
b. polysorbate 80 (PS80); and
c. diethylenetriaminepentaacetic acid (DTPA).

6. The formulation of activatable antibody of claim 5 comprising:
a. 20 mM histidine (pH 5.5);
b. 0.05% PS80; and
c. 50 M DTPA.

7. The formulation of activatable antibody of claim 6 comprising 50 mg/ml Activatable Ipilimumab.

8. The formulation of activatable antibody of claim 6 comprising 80 mg/ml Activatable Ipilimumab.

9-38. (canceled)

39. A method of making a lyophilized unit dose formulation of an activatable antibody comprising:
a. providing a formulation comprising:
i. approximately 83 mM sucrose; and
ii. approximately 313 mM mannitol or 760 mM glycine; and
b. lyophilizing the formulation in a process that includes an annealing step.

40. The method of claim 39 wherein the activatable antibody comprises a cleavable moiety comprising the sequence of SEQ ID NO: 19.

41. The method of claim 40 wherein the activatable antibody is Activatable Ipilimumab comprising:
c. a heavy chain comprising the heavy chain variable region sequence of SEQ ID NO: 9; and
d. a light chain comprising the light chain variable region sequence of SEQ ID NO: 22.

42. The method of claim 41 wherein the annealing step comprises annealing for 3 hours or 5 hours.

43. The method of claim 41 further comprising chilling the filled vials to 5° C. and holding them for 2 h followed by chilling the filled vials to −5° C. for 2 h.

44. The method of claim 39 further comprising sealing the lyophilized unit dose formulation in a vial under vacuum.

45. The method of claim 44 wherein the vacuum is approximately 500 mTorr.
